(19) United States
(12) Patent Application Publication
(10) Pub. No.: US 2026/0240587 A1
(43) Pub. Date: Aug. 20, 2026

(54) ELECTRODE CARTRIDGE FOR SKIN TREATMENT AND ELECTRODE CARTRIDGE COUPLING STRUCTURE COMPRISING SAME

(71) Applicant: CLASSYS INC., Seoul (KR)

(72) Inventors: Dae Uk Yim, Hwaseong-si, Gyeonggi-do (KR); Sung Ho Park, Gunpo-si, Gyeonggi-do (KR); Hye Jin Kim, Seoul (KR)

(73) Assignee: CLASSYS INC., Seoul (KR)

(21) Appl. No.: 19/480,160

(22) PCT Filed: Apr. 30, 2024
(86) PCT No.: PCT/KR2024/005853

§ 371 Date: Oct. 30, 2025

(30) Foreign Application Priority Data
May 3, 2023 (KR) 10-2023-0057462

(51) Int. Cl. A61B 18/12; A61B 18/00; A61H 7/00; A61M 37/00

(52) U.S. Cl. CPC A61B 18/1206; A61H 7/002; A61M 37/0015; A61B 2018/00178; A61B 2018/00202; A61B 2018/00452; A61B 2018/0094; A61B 2018/126; A61H 2201/0153; A61H 2201/105; A61M 2037/0023; A61M 2037/0046; A61M 2205/054

(57) ABSTRACT

The present invention relates to an electrode cartridge for skin treatment and an electrode cartridge coupling structure comprising same, comprising: an electrode unit which is contacted to skin so as to apply electrical stimulation thereto; and a drug supply unit which is separably coupled to the electrode unit and is contacted to skin so as to apply a drug thereon or infiltrate the drug thereinto. The drug supply unit for applying the drug on skin or infiltrating the drug thereinto, and the electrode for applying the electrical stimulation are together contacted to skin and rotated, and thus a skin care and treatment effect is greatly enhanced, and the drug supply unit for applying the drug on skin or infiltrating the drug thereinto is separably coupled to the electrode unit for applying the electrical stimulation, and thus the drug supply unit can be easily replaced after a procedure.

### TECHNICAL FIELD

The present disclosure relates to an electrode cartridge coupling structure and, more particularly, to an electrode cartridge for skin treatment and an electrode cartridge coupling structure including the same, configured to provide ease of periodic cartridge replacement and to improve the efficacy of skin treatment and care.

### BACKGROUND ART

response to increasing interest in skin beauty, a variety of skin treatment devices have been developed.

Skin treatment devices have been proposed in various configurations, such as devices utilizing ultrasound, laser irradiation, or radio frequency (RF) electrodes.

Skin treatment devices that use ultrasound includes an ultrasound treatment handpiece that contacts the skin of a subject to be treated and emits ultrasound waves into the skin, and a main body that controls the operation of the ultrasound treatment handpiece.

Ultrasound-based skin treatment devices are primarily used for procedures that intentionally target and damage specific skin tissues or break down fat tissue by employing high-intensity focused ultrasound (HIFU) to create thermal lesions at a specific depth beneath the skin's surface using an ultrasound treatment handpiece.

Laser skin treatment devices are used for various purposes such as hair loss prevention, hair growth promotion, skin peeling and regeneration, skin brightening and whitening, removal of wrinkles, spots, and freckles. The devices emit a beam of a certain size, called a spot size, onto the skin, and the treatment involves applying laser-generated heat only to the target area to thermally damage the skin tissue.

RF electrode skin treatment devices are utilized for diverse dermatological applications. These applications include skin remodeling and resurfacing achieved by heating the lower dermis to contract existing collagen and trigger a wound-healing response, as well as wrinkle removal, and treatment of sebaceous glands, hair follicles, fatty tissue, and spider veins.

A skin treatment device is tool that uses a handpiece with a detachable cartridge to perform skin treatments. The user (therapist) holds and places the cartridge directly on the skin, and then operates the cartridge to deliver the treatment.

The cartridge has a preset number of uses and needs to be replaced after the preset number of uses.

A cartridge for a skin treatment device, including ultrasound, laser, and RF electrode types, is configured to electrically connect to a handpiece upon attachment, and the operation of the cartridge is controlled by receiving electric power from a control unit.

A conventional cartridge for a skin treatment device has the disadvantage of being complex in structure and requiring high manufacturing costs, as the cartridge is equipped with a locking structure and an electrical contact structure separately to maintain the joined state with a handpiece after attachment and to be electrically connected to a control unit through the connection with the handpiece.

In addition, if the handpiece is structured to be electrically connected to the cartridge via a wire upon engagement, this may result in various restrictions and inconveniences during cartridge attachment and detachment, as well as during cleaning.

In particular, in the case of RF cartridges that treat the skin through rotational movement while maintaining contact between the skin and RF electrodes, the electrical connection structure is complex, leading to inconvenience in connecting and disconnecting the cartridge from the handpiece during replacement.

In addition, conventional cartridges for skin treatment devices are limited in their effectiveness for skin care and treatment, as the procedure is performed solely with the electrode in contact with the skin.

Korean Patent No. 10-2206623 “NEEDLE TIP MOUNTED ON SKIN CARE DEVICE AND SKIN CARE DEVICE” discloses a structure including a needle electrode and an application pad for delivering a drug to the skin.

However, Korean Patent No. 10-2206623 “NEEDLE TIP MOUNTED ON SKIN CARE DEVICE AND SKIN CARE DEVICE” does not provide a massage effect on the skin, and only applies simple electrical stimulation, limiting its skin care and treatment effects. This is because the device is structured to move needle electrodes in a vertical reciprocating motion, allowing the needle electrodes to penetrate the skin and apply electrical stimulation.

Moreover, in Korean Patent No. 10-2206623 “NEEDLE TIP MOUNTED ON SKIN CARE DEVICE AND SKIN CARE DEVICE”, the application pad is placed on the bottom of a housing to apply the drug. When treating multiple patients, the drug needs to be reapplied for each session, which raises hygiene issues. Additionally, it is inconvenient to remove any remaining drug after each use and reapply a new dose.

### DISCLOSURE

### Technical Problem

An objective of the present disclosure is to provide an electrode cartridge for skin treatment and an electrode cartridge coupling structure including the same, which can enhance skin care and treatment effects by simultaneously bringing a drug supply part that applies or invasively delivers a drug to the skin and an electrode that provides electrical stimulation into contact with the skin, while rotating both components together.

An objective of the present disclosure is to provide an electrode cartridge for skin treatment and an electrode cartridge coupling structure including the same, in which a drug supply part that applies or invasively delivers a drug to the skin is detachably connected to an electrode that provides electrical stimulation, making it easy to replace the drug supply part after treatment.

An objective of the present disclosure is to provide an electrode cartridge for skin treatment and an electrode cartridge coupling structure including the same, which can provide ease of periodic cartridge replacement and can simplify the electrical connection structure.

### Technical Solution

In order to achieve the above mentioned objectives, according to an embodiment of the present disclosure, there is provided an electrode cartridge for skin treatment, the cartridge including: an electrode part disposed to contact a skin and provide electrical stimulation thereto; and a drug supply part detachably coupled to the electrode part and configured to apply or invasively deliver a drug to the skin.

In the present disclosure, the drug supply part may be a drug supply roll member formed in a roll shape and equipped with multiple microneedles on an outer surface thereof for transdermal drug delivery.

In the present disclosure, the drug supply roll member may be configured such that the microneedles are made from a biodegradable material containing a drug ingredient so that when the microneedles penetrate the skin, the microneedles may dissolve through interaction with body fluids, releasing the drug ingredient contained within into the skin.

In the present disclosure, the electrode part may include a first electrode panel member and a second electrode panel member respectively provided on opposite sides of the drug supply roll member and detachably connected to each other by penetrating the drug supply roll member.

In the present disclosure, the first electrode panel member and the second electrode panel member may each have massage protrusions, which can stimulate the skin, spaced apart from each other on a contact surface that comes into contact with the skin.

In the present disclosure, the drug supply roll member has an electrode-coupling hollow part disposed in a width direction in a manner that enables penetration, wherein the first electrode panel member may have a first electrode coupling part protruding from a surface thereof facing the second electrode panel member to be inserted into the electrode-coupling hollow part, and the second electrode panel member may have a second electrode coupling part protruding from a surface thereof facing the first electrode panel member to be inserted into the electrode-coupling hollow part and detachably coupled to the first electrode coupling part.

In the present disclosure, the first electrode coupling part and the second electrode coupling part may be detachably connected to each other by screw connection.

In addition, in order to achieve the above mentioned objectives, according to an embodiment of the present disclosure, there is provided an electrode cartridge coupling structure including: an electrode cartridge including an electrode part disposed to contact a patient's skin and apply an electric signal thereto; and a cartridge coupling object provided with a cartridge coupling part into which a portion of the electrode cartridge is inserted and joined, and electrically connected to a control body that controls an operation of the electrode cartridge, wherein the electrode cartridge may include an embodiment of an electrode cartridge for skin treatment according to the present disclosure.

An embodiment of the electrode cartridge coupling structure according to the present disclosure may further include: a ball plunger part protruding from each side of the electrode cartridge; and a ball insertion part provided on each inner side of the cartridge coupling part and into which the ball plunger part is inserted, wherein the electrode cartridge is positioned so as to be rotatable around the ball plunger parts.

In the present disclosure, the ball plunger part and the ball insertion part may share an electrical terminal structure that provides electrical connectivity therebetween so as to electrically connect the electrode part and the control body.

An embodiment of the electrode cartridge coupling structure according to the present disclosure may further include an auxiliary electrode part located on an end side of the cartridge coupling part, wherein the electrode part may function as an RF electrode having one polarity, and the auxiliary electrode part may function as an RF electrode having the opposite polarity.

In the present disclosure, the electrode cartridge may be provided with a ring-shaped contact protrusion on each side thereof that surrounds the ball plunger part.

In the present disclosure, on the inner surface of the cartridge coupling part, a cartridge stopper part may be positioned to protrude, which catches the contact protrusion and prevents the electrode cartridge from being pushed and moved inward of the cartridge coupling part while in contact with the skin.

In the present disclosure, the cartridge stopper part may be provided with a ball bearing part that rolls while in contact with the contact protrusion on the surface thereof facing the contact protrusion.

An embodiment of the electrode cartridge coupling structure according to the present disclosure may further include a cartridge separation part positioned within the cartridge coupling part to be able to move forward and backward in a coupling direction of the electrode cartridge, and configured to push the electrode cartridge to separate the electrode cartridge from the cartridge coupling part.

In the present disclosure, the cartridge separation part may include: a pusher member positioned to be able to move forward and backward within the cartridge coupling part; and a linear movement device positioned in the cartridge coupling object, and configured to move the pusher member forward and backward.

In the present disclosure, the cartridge separation part may further include a manipulation switch part located on the cartridge coupling object to allow a practitioner to control an operation of the linear movement device.

### Advantageous Effects

According to the present disclosure, the drug supply part that applies or invasively delivers a drug to the skin and the electrode that provides electrical stimulation are brought into contact with the skin and rotated together, thereby greatly improving the skin care and treatment effect.

According to the present disclosure, the drug supply part that applies or invasively delivers a drug to the skin is detachably connected to the electrode part that provides electrical stimulation, so that the drug supply part can be easily replaced after the procedure, enabling hygienic use and greatly improving the satisfaction with the procedure.

According to the present disclosure, ease of periodic cartridge replacement can be provided and the electrical connection structure can be simplified, thereby improving convenience of treatment and reducing manufacturing costs.

### DESCRIPTION OF DRAWINGS

FIG. 1 is a perspective view showing an embodiment of an electrode cartridge coupling structure according to the present disclosure.

FIG. 2 is an exploded perspective view showing an embodiment of an electrode cartridge coupling structure according to the present disclosure.

FIG. 3 is a partial cross-sectional view showing an embodiment of an electrode cartridge coupling structure according to the present disclosure.

FIG. 4 is a plan view showing another embodiment of an electrode cartridge coupling structure according to the present disclosure.

### DESCRIPTION OF REFERENCE NUMERALS

100: electrode cartridge 100a: contact protrusion
110: electrode part 111: first electrode panel member
111a: first electrode coupling part 112: second electrode panel member
112a: second electrode coupling part 113: massage protrusion
120: drug supply part 121: drug supply roll member
121a: microneedle 121b: electrode-coupling hollow part
200: ball plunger part 210: ball member
220: spring member 300: cartridge coupling object
300a: handpiece body 300b: auxiliary electrode part
310: cartridge coupling part 320: ball insertion part
321: ball insertion terminal member 330: cartridge stopper part
340: ball bearing part 400: cartridge separation part
410: pusher member 420: linear movement device
430: manipulation switch part 431: forward switch
432: reverse switch

### BEST MODE

Hereinafter, the present disclosure will be described in more detail.

Hereinafter, a preferred embodiment of the present disclosure will be described in detail with reference to the attached drawings. Before proceeding with a detailed description of the present disclosure, it should be noted that the terms and words used in the specification and claims described below should not be construed as limited to their conventional or dictionary meanings. Therefore, it should be understood that the embodiments described in this specification and the configurations illustrated in the drawings are only the most preferred embodiments of the present disclosure and do not represent all of the technical ideas of the present disclosure, and that there may be various equivalents and modified examples that can replace them at the time of filing this application.

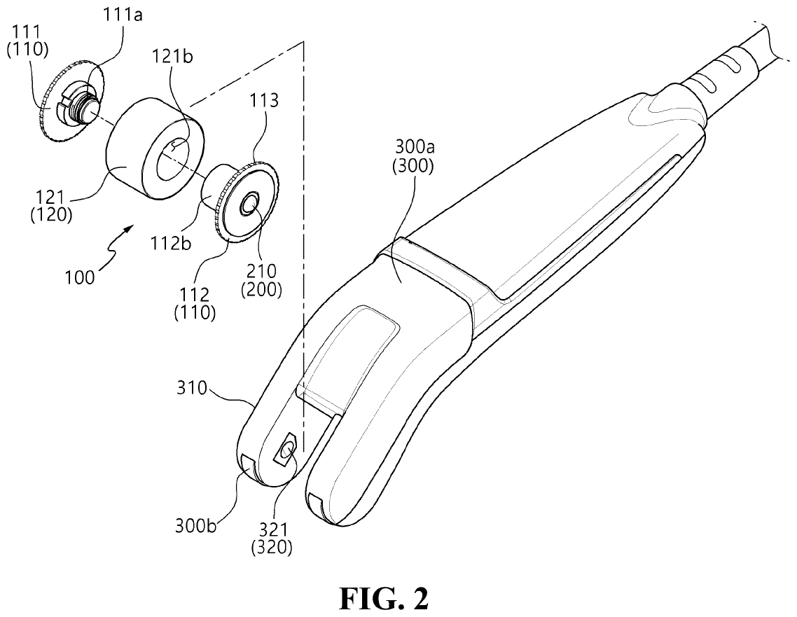

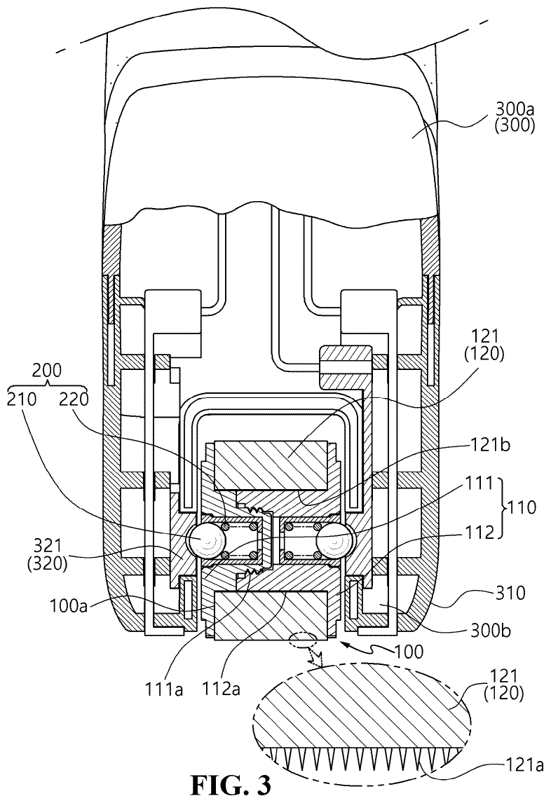

FIG. 2 is an exploded perspective view showing an embodiment of an electrode cartridge coupling structure according to the present disclosure, FIG. 2 is an exploded perspective view showing an embodiment of an electrode cartridge coupling structure according to the present disclosure, and FIG. 3 is a partial cross-sectional view showing an embodiment of an electrode cartridge coupling structure according to the present disclosure.

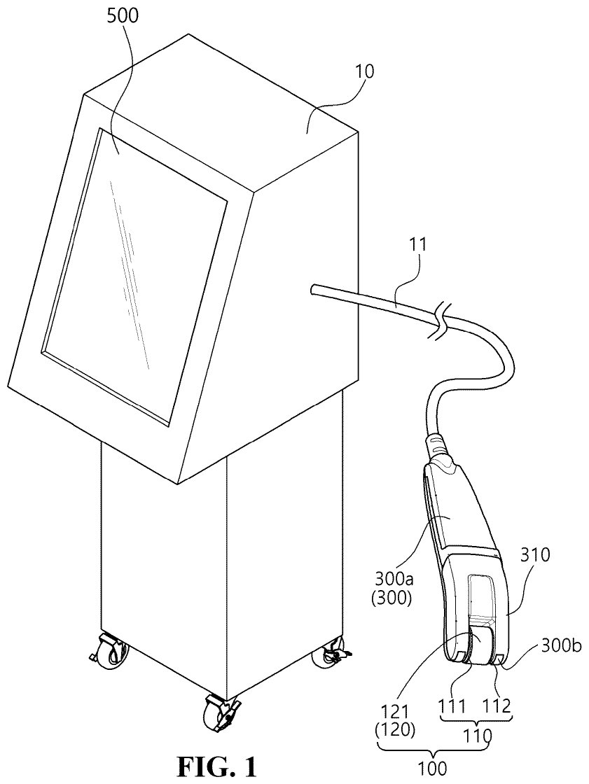

Referring to FIGS. 1 to 3, an embodiment of an electrode cartridge coupling structure according to the present disclosure is described in detail below.

An embodiment of an electrode cartridge coupling structure according to the present disclosure includes an electrode cartridge 100 including an electrode part 110 that comes into contact with the patient's skin and applies an electric signal to the patient's skin.

In addition, an embodiment of an electrode cartridge coupling structure according to the present disclosure includes a cartridge coupling object 300 provided with a cartridge coupling part 310 into which a portion of the electrode cartridge 100 is inserted and joined.

The cartridge coupling part 310 has a structure in which a cartridge insertion part into which the electrode cartridge 100 can be inserted is located between the cartridge coupling parts 310 on the end side of the cartridge coupling object 300. The cartridge insertion part is formed in an open shape toward the end of the cartridge coupling object 300 between the cartridge coupling parts 310, and the electrode cartridge 100 may be inserted into the cartridge insertion part between the cartridge coupling parts 310 and detachably coupled.

The electrode cartridge 100 includes the electrode part 110 that receives electric power and applies an electric signal. The electrode part 110 is electrically connected to the cartridge coupling object 300 when the electrode cartridge 100 is coupled to the cartridge coupling object 300, and is operated by receiving electric power from the cartridge object 300.

The electrode part 110 is an RF electrode that generates an RF signal, for example, and may be modified and implemented in various ways, including a known electrode that can contact the patient's skin and provide stimulation with an electric signal.

Meanwhile, the cartridge coupling object 300 is, as an example, a handpiece body 300a to which the electrode cartridge 100 is detachably coupled, which is held by the practitioner's hand and used, and which electrically connects the electrode part 110 to a control body 10.

The handpiece body 300a is connected to the control body 10 by a connection cable 11. The control body 10 includes a controller that is electrically connected to the electrode part 110 of the electrode cartridge 100 through the handpiece body 300a and controls the operation of the electrode part 110.

The control body 10 is known to control the operation of the handpiece body 300a to which the electrode cartridge 100 having the electrode part 110 that comes into contact with the patient's skin is coupled, and can be modified in various ways, and thus a more detailed description thereof will be omitted.

The control body 10 may include may include a display panel 10a that outputs the control mode of the handpiece body 300a to the screen so as to control the operation of each handpiece body 300a, and the display panel 10a is, as an example, a touch screen panel that allows input of operation control signals by touching the output screen.

The operation control mode of each handpiece body 300a and inputting an operation control signal to activate the operation control mode are known in skin treatment devices, and thus a detailed description thereof is omitted.

Meanwhile, the electrode cartridge 100 is detachably coupled to the cartridge coupling object 300, that is, the handpiece body 300a.

In addition, the electrode cartridge includes: the electrode part 110 that contacts the skin to deliver electrical stimulation; and a drug supply part 120 that is detachably connected to the electrode part 110 and applies or invasively delivers drug to the skin.

The electrode cartridge 100 is rotatably coupled to the cartridge coupling object 300, that is, the handpiece body 300a, and is attached in a detachable manner.

The electrode cartridge 100 includes the drug supply part 120 and the electrode part 110 that come into contact with the patient's skin. The electrode part 110 and the drug supply part 120 rotate while in contact with the skin to evenly deliver the drug to the skin and stably apply the RF signal.

To be specific, the drug supply part 120 is, as an example, a drug supply roll member 121 formed in a roll shape and equipped with multiple microneedles 121a on the outer surface thereof for transdermal drug delivery.

The drug supply roll member 121 has the multiple microneedles 121a spaced apart on the outer surface thereof, which penetrate the skin and deliver drugs containing active ingredients effective for wrinkle reduction, hydration, and improved elasticity.

The drug supply roll member 121 may be configured such that drug ingredients are coated on the surface of the microneedles 121a, and when the microneedles penetrate the skin, the coated drug ingredients dissolve in the body fluids and are delivered into the skin.

Alternatively, the drug supply roll member 121 may be configured such that the microneedles 121a are made from a biodegradable material containing drug ingredients so that when the microneedles penetrate the skin, the microneedles 121a dissolve through interaction with body fluids, releasing the drug ingredients contained within into the skin.

Alternatively, the drug supply roll member 121 may be configured such that a hollow space filled with drug ingredients is formed inside the microneedles, and when the microneedles penetrate the skin, the drug ingredients coated on the microneedles dissolve in the body fluids and are delivered into the skin.

In the present disclosure, as an example, the drug supply roll member 121 is configured such that drug ingredients are coated on the surface of the microneedles 121a, and when the microneedles penetrate the skin, the coated drug ingredients dissolve in the body fluids and are delivered into the skin, or is configured such that the microneedles 121a are made from a biodegradable material containing drug ingredients so that when the microneedles penetrate the skin, the microneedles 121a dissolve through interaction with body fluids, releasing the drug ingredients contained within into the skin.

The drug that is delivered into the skin by means of the drug supply part 120, that is, the drug supply roll member 121, is a skin-enhancing formulation containing well-known active ingredients that reduce wrinkles and enhance moisture and elasticity, and a detailed description is omitted.

The drug supply roll member 121 may be separated from the electrode part 110 that comes into contact with the skin, and may be used once per patient, allowing for hygienic use.

The electrode part 110 includes a first electrode panel member 111 and a second electrode panel member 112 respectively provided on opposite sides of the drug supply roll member 121 and detachably connected to each other by penetrating the drug supply roll member 121.

As an example, the first electrode panel member 111 and the second electrode panel member 112 are formed in a circular shape. The first electrode panel member 111 and the second electrode panel member 112 may have a shape in which the contact surface that comes into contact with the skin is flat, or may have a shape in which a plurality of massage protrusions 113 that can stimulate the skin is provided spaced apart on the contact surface that comes into contact with the skin.

The electrode part 110 may have the contact surface that comes into contact with the skin formed in various shapes, so that an electrode cartridge including an electrode part 110 having a shape suitable for the treatment may be selectively coupled with the cartridge coupling object 300 according to the type of skin treatment.

To be specific, in the drug supply roll member 121, an electrode-coupling hollow part 121b is disposed in the width direction in a manner that enables penetration. The first electrode panel member 111 has a first electrode coupling part 111a protruding from one surface thereof facing the second electrode panel member 112 to be inserted into the electrode-coupling hollow part 121b, and the second electrode panel member 112 has a second electrode coupling part 112a protruding from one surface thereof facing the first electrode panel member 111 to be inserted into the electrode-coupling hollow part 121b and detachably coupled to the first electrode coupling part 111a.

The first electrode coupling part 111a and the second electrode coupling part 112a are structured to be coupled and electrically connected to each other, so that when an electrical connection is made to only one of the first electrode coupling part 111a and the second electrode coupling part 112a, electric power is applied to each of the first electrode coupling part 111a and the second electrode coupling part 112a.

As an example, the first electrode coupling part 111a and the second electrode coupling part 112a are detachably connected to each other by screw connection. In addition, various combinations may be performed using a known detachable coupling structure such as a forced-fit connection.

The first electrode coupling part 111a has a protruding bolt part, and the second electrode coupling part 112a has a screw groove part into which the bolt part is inserted and screw-coupled.

The first electrode coupling part 111a and the second electrode coupling part 112a are screw-coupled to allow easy assembly and disassembly, while ensuring a secure connection when joined.

The electrode cartridge 100 according to the present disclosure is detachably coupled to the cartridge coupling part 310 located at the end side of the cartridge coupling object 300 and may be easily replaced.

The first electrode panel member 111 and the second electrode panel member 112 are assembled in a state in which the first electrode coupling part 111a and the second electrode coupling part 112a are inserted into the electrode-coupling hollow part 121b on opposite sides of the drug supply roll member 121 by screw connection and are in close contact with each other on opposite sides of the drug supply roll member 121.

Once the first electrode coupling part 111a and the second electrode coupling part 112a are separated, the drug supply roll member 121 may be easily separated from the electrode part 110, that is, the first electrode panel member 111 and the second electrode panel member 112.

The electrode cartridge 100 of the present disclosure including the electrode part 110 and the drug supply roll member 121 is inserted between the pair of cartridge coupling parts 310 protruding from the end side of the cartridge coupling object 300 and is detachably coupled to the cartridge coupling object 300.

The electrode cartridge 100 is positioned so that a portion protrudes toward the end of the cartridge coupling part 310 so as to come into contact with the patient's skin. On opposite sides of the electrode cartridge 100, the cartridge coupling parts 310 are provided for detachably coupling the electrode cartridge 100 to the cartridge coupling object.

The cartridge coupling part 310 includes: a ball plunger part 200 protruding from each side of the electrode cartridge 100; and a ball insertion part 320 provided on each inner side of the cartridge coupling part 310 and into which the ball plunger part 200 is inserted.

The ball plunger part 200 includes: a ball member 210 protruding from each side of the electrode cartridge 100, that is, from a portion of the first electrode panel member 111 and a portion of the second electrode panel member 112; and a spring member 220 located inside the electrode cartridge 100 and elastically supporting the ball member 210.

The ball plunger part 200 is inserted into the ball insertion part 320 on the inner side of the cartridge coupling part 310 as the ball members 210 protruding partly from the sides of the electrode cartridge 100 are elastically supported by the spring members 220, so that a portion of the electrode cartridge 100 is coupled to the cartridge coupling object 300, that is, mounted between the pair of the cartridge coupling parts 310 of the handpiece body 300a.

The electrode cartridge 100 is securely retained in a coupled state with the handpiece body 300a when a portion of the ball member 210 of the ball plunger part 200 is inserted into the ball insertion part 320 provided on each inner side of the cartridge coupling part 310. The electrode cartridge 100 may be easily separated from the handpiece body 300a when the ball member 210 is subjected to a force in the direction opposite to the coupling direction, causing the ball member 210 to be pulled out of the ball insertion part 320.

That is, the electrode cartridge 100 may be prepared for skin treatment by simply inserting and pushing a portion of the electrode cartridge 100 between the cartridge coupling parts 310 so that the ball plunger part 200 is inserted into the ball insertion part 320, thereby connecting the electrode cartridge 100 to the handpiece body 300a. When the lifespan of the electrode cartridge 100 ends or a malfunctions occurs, the electrode cartridge 100 may be easily detached, which improves user convenience during replacement.

In addition, the electrode part 110 of the electrode cartridge 100 is an RF electrode, as an example, and may apply an RF high-frequency signal in a monopolar configuration using a single active polarity and a separate ground electrode.

An embodiment of the electrode cartridge coupling structure according to the present disclosure further includes an auxiliary electrode part 300b located on the end side of the cartridge coupling part 310.

While the electrode part 110 of the electrode cartridge 100 functions as an RF electrode having one polarity, the auxiliary electrode part 300b located on the end side of the cartridge coupling part 310 functions as an RF electrode having the opposite polarity, enabling the application of an RF high-frequency signal in a bipolar configuration.

The auxiliary electrode part 300b is electrically connected to the control body 10 by a wire or cable wired inside the cartridge coupling object 300.

Meanwhile, the ball plunger part 200 and the ball insertion part 320 share an electrical terminal structure that provides electrical connectivity therebetween so as to electrically connect the electrode part 110 of the electrode cartridge 100 and the cartridge coupling object 300, that is, the control body 10, thereby supplying electric power to the electrode part 110 of the electrode cartridge 100 and controlling the operation of the electrode part 110.

To be specific, the ball insertion part 320 has a ball insertion groove and is electrically connected to the control body 10 via a wire or cable due to a ball insertion terminal member 321 made of an electrically conductive material. The ball plunger part 200 provided on each of the first electrode panel member 111 and the second electrode panel member 112 is electrically connected to the ball insertion terminal member 321 while the ball members 210 elastically supported by the spring members 220 are maintained in a state of being connected to the first electrode panel member 111 and the second electrode panel member 112, respectively, so that electric power may be supplied to the first electrode panel member 111 and the second electrode panel member 112.

The ball insertion terminal member 321 is electrically connected to the control body 10 via the connection cable 11 of the handpiece body 300a.

Thus, when the electrode cartridge 100 is mounted into the cartridge coupling parts 310 of the handpiece body 300a, the ball plunger parts 200 are inserted into the ball insertion grooves of the ball insertion terminal members 321, and the electrode part 110 is electrically connected to the control body 10 via the connection cable 11 of the handpiece body 300a, so that the operation of the electrode part 110 may be controlled by means of the control body 10.

The structure in which the electrode cartridge 100 coupled to the handpiece body 300a is electrically connected to the control body 10 through the connection cable 11 is a known structure and can be implemented in various ways, and thus a more detailed description thereof is omitted.

The electrode cartridge 100 is provided with a ring-shaped contact protrusion 100a on each side thereof that surrounds the ball plunger part 200, and the contact protrusion 100a has a width of less than 30% of the diameter of the ball member 210 so as to minimize the contact area between the both sides of the electrode cartridge 100 and the inner surfaces of the cartridge coupling parts 310.

When the electrode cartridge 100 is coupled to or separated from the cartridge coupling parts 310, since only the contact protrusions 100a come into contact with the inner surfaces of the cartridge coupling parts 310, friction may be minimized and the force required for coupling to or separating from the handpiece body 300a may be minimized.

To be specific, the electrode cartridge 100 may be rotated around the ball plunger parts 200 with the ball plunger parts 200 protruding on opposite sides inserted into the ball insertion parts.

In addition, as an example, the electrode cartridge 100 is a cartridge for RF treatment applications, which rotates while the ball plunger parts 200 positioned on the outer surfaces of the first electrode panel member 111 and the second electrode panel member 112, respectively, are inserted into the ball insertion parts 320, and RF high-frequency energy is applied between the first electrode panel member 111 and the auxiliary electrode part 300b and between the second electrode panel member 112 and the auxiliary electrode part 300b.

In the electrode cartridge 100, that is, in the first electrode panel member 111 and the second electrode panel member 112, the ring-shaped contact protrusions 100a protrude, which come into contact with the inner surfaces of the cartridge coupling parts 310 and surround the ball plunger parts 200.

The contact protrusions 100a are ring-shaped protrusions that surround the ball plunger parts 200 and minimize the contact area between the outer surfaces of the first electrode panel member 111 and the second electrode panel member 112 and the inner surfaces of the cartridge coupling parts 310.

Of the outer surfaces of the first electrode panel member 111 and the second electrode panel member 112, since only the contact protrusions 100a come into contact with the inner surfaces of the cartridge coupling parts 310, the electrode cartridge 100 may rotate smoothly around the ball plunger parts 200.

The electrode cartridge 100 may rotate around the ball plunger parts 200 as the ball plunger parts 200 are inserted into the ball insertion parts 320, and even when the electrode cartridge 100 rotates around the ball plunger parts 200, the ball members being elastically supported are stably connected to the ball insertion parts 320, so that electric power may be stably supplied to the first electrode panel member 111 and the second electrode panel member 112.

A practitioner holds the handpiece body 300a and performs a treatment by applying RF signals to the patient's skin by pushing or pulling the handpiece body 300a to rotate the electrode cartridge 100 while the electrode part 110 of the electrode cartridge 100 and the drug supply roll member 121 are in contact with the skin, and delivering a drug to the treatment site through microneedles 121a of the drug supply roll member 121.

At this time, the electrode cartridge 100 rotates around the ball plunger parts 200, and the ball members of the ball plunger parts 200 are securely retained in an electrically connected state within the ball insertion parts 320, thereby enabling stable application of RF signals to the skin.

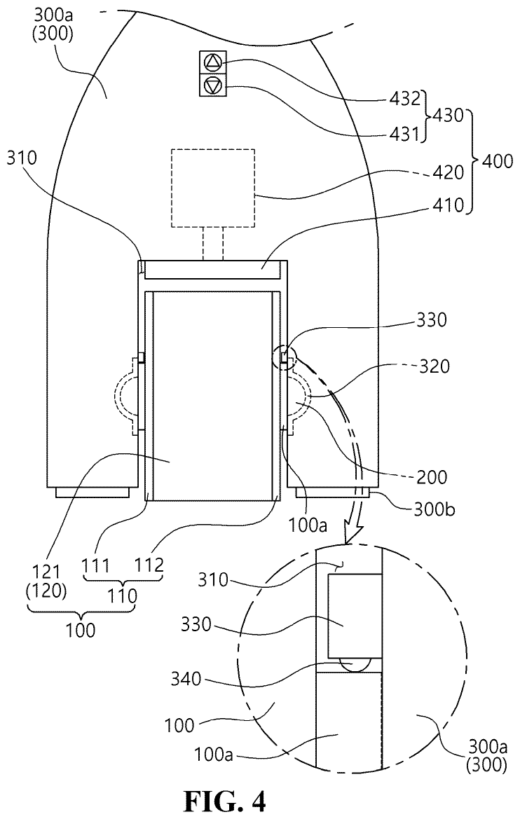

Meanwhile, FIG. 4 is a plan view showing another embodiment of an electrode cartridge coupling structure according to the present disclosure. Referring to FIG. 4, in another embodiment of an electrode cartridge coupling structure according to the present disclosure, on the inner surface of the cartridge coupling part 310, a cartridge stopper part 330 is positioned to protrude, which catches the contact protrusion 100a and prevents the electrode cartridge 100 from being pushed and moved inward of the cartridge coupling part 310 while in contact with the skin.

The cartridge stopper part 330 is positioned so as to protrude from the inner side of the ball insertion part 320 on the inner surface of the cartridge coupling part 310, so that the contact protrusion 100a protruding from the outer surface of the electrode cartridge 100 is caught by the cartridge stopper part 330 when the electrode cartridge 100 in contact with the skin is pushed inward into the cartridge coupling part 310.

The electrode cartridge 100 is connected to the handpiece body 300a by means of the ball plunger parts 200, and the practitioner may perform the procedure by holding and pressing the handpiece body 300a while the electrode cartridge 100 is in contact with the skin.

When the practitioner holds the handpiece body 300a and presses the electrode cartridge 100 being in contact with the skin, the electrode cartridge 100 is pushed inward into the cartridge coupling part 310, so that the ball plunger parts 200 may be separated from the ball insertion parts 320.

The cartridge stopper part 330 prevents the electrode cartridge 100 from being pushed inward into the cartridge coupling part 310 as the contact protrusion 100a is caught on the cartridge stopper part 330 when the electrode cartridge 100 in contact with the skin is pressed by the practitioner, and keeps the ball plunger parts 200 stably inserted within the ball insertion parts 320.

In addition, the cartridge stopper part 330 is provided with a ball bearing part 340 that rolls while in contact with the contact protrusion 100a on the surface thereof facing the contact protrusion 100a to facilitate smooth rotational movement of the electrode cartridge 100.

The electrode cartridge 100 is rotated around the ball plunger parts 200 while remaining in contact with the skin during the treatment of the patient's skin. However, the cartridge 100 may not rotate smoothly due to friction caused by the contact protrusion 100a coming into contact with the cartridge stopper part 330.

The ball bearing part 340 rolls in contact with the contact protrusion 100a when the electrode cartridge 100 rotates while remaining in contact with the skin, thereby facilitating smooth rotational movement of the electrode cartridge 100.

Thus, during the procedure, the electrode cartridge 100 rotates smoothly while in contact with the skin, and even when excessive pressure is applied to the electrode cartridge 100 against the skin, the ball plunger parts 200 do not come off from the ball insertion parts 320, enabling stable treatment and greatly improving practitioner convenience.

In addition, another embodiment of an electrode cartridge coupling structure according to the present disclosure may further include a cartridge separation part 400 positioned within the cartridge coupling parts 310 to be able to move forward and backward in the coupling direction of the electrode cartridge 100, and configured to push the electrode cartridge 100 to separate the electrode cartridge 100 from the cartridge coupling parts 310.

The cartridge separation part 400 includes: a pusher member 410 positioned to be able to move forward and backward in the coupling direction of the electrode cartridge 100 within the cartridge coupling parts 310; and a linear movement device 420 positioned within the cartridge coupling object 300, that is, the handpiece body 300a, and configured to move the pusher member 410 forward and backward.

In addition, the cartridge separation part 400 may further include a manipulation switch part 430 located on the handpiece body 300a to allow the practitioner to control the operation of the linear movement device 420.

The manipulation switch part 430 includes a forward switch 431 and a reverse switch 432. The practitioner may press the forward switch 431 to advance the pusher member 410 by means of the linear movement device 420 and push the electrode cartridge 100 outward from the cartridge coupling parts 310 with the pusher member 410 to separate the electrode cartridge 100 from the handpiece body 300a.

In addition, when the practitioner presses the reverse switch 432, the linear movement device 420 moves the pusher member 410 backward to return to the original position thereof.

The linear movement device 420 is, as an example, a ball screw-type linear actuator. The linear movement device 420 may be implemented in various ways by using a rack and pinion mechanism for converting the motor's rotational power into linear motion, including a rack gear and a pinion gear that is meshed with the rack gear and rotated by the motor, a hydraulic cylinder, and other known lifting and lowering devices, and thus a more detailed description is omitted.

Although the electrode cartridge 100 is structured to be coupled to the handpiece body 300a, it may be difficult for the practitioner to hold, and when the practitioner holds the electrode cartridge 100 with the electrode part 110 positioned on the outer surface thereof with his or her fingers to separate the electrode cartridge 100 from the handpiece body 300a, there is a risk of finger burns if the electrode part 110 is in an energized state, that is, when an RF signal is applied.

The cartridge separation part 400 enables easy and safe separation of the electrode cartridge 100 from the cartridge coupling object 300, that is, the handpiece body 300a, by pushing the electrode cartridge 100 with the pusher member 410 upon replacement of the electrode cartridge 100.

The present disclosure may greatly improve the skin care and treatment effect since the drug supply part 120 that applies or invasively delivers a drug to the skin and the electrode that provides electrical stimulation are brought into contact with the skin and rotated together.

The present disclosure may enable hygienic use and greatly improve the satisfaction with the procedure since the drug supply part 120 that applies or invasively delivers a drug to the skin is detachably connected to the electrode part that provides electrical stimulation, so that the drug supply part 120 can be easily replaced after the procedure.

The present disclosure may improve convenience of treatment and reduce manufacturing costs since ease of periodic cartridge replacement may be provided and the electrical connection structure may be simplified.

It is to be understood that the present disclosure is not limited to the above-described embodiments, and can be implemented by making various changes without departing from the spirit of the present disclosure, and these changes are also included in the composition of the present disclosure.

1: An electrode cartridge for skin treatment, the cartridge comprising:
an electrode part disposed to contact a skin and provide electrical stimulation thereto; and
a drug supply part detachably coupled to the electrode part and configured to apply or invasively deliver a drug to the skin.

2: The cartridge of claim 1, wherein the drug supply part is a drug supply roll member formed in a roll shape and equipped with multiple microneedles on an outer surface thereof for transdermal drug delivery.

3: The cartridge of claim 2, wherein the drug supply roll member is configured such that the microneedles are made from a biodegradable material containing a drug ingredient so that when the microneedles penetrate the skin, the microneedles dissolve through interaction with body fluids, releasing the drug ingredient contained within into the skin.

4: The cartridge of claim 2, wherein the electrode part comprises:
a first electrode panel member and a second electrode panel member respectively provided on opposite sides of the drug supply roll member and detachably connected to each other by penetrating the drug supply roll member.

5: The cartridge of claim 4, wherein the first electrode panel member and the second electrode panel member each have massage protrusions, which can stimulate the skin, spaced apart from each other on a contact surface that comes into contact with the skin.

6: The cartridge of claim 4, wherein the drug supply roll member has an electrode-coupling hollow part disposed in a width direction in a manner that enables penetration,
wherein the first electrode panel member has a first electrode coupling part protruding from a surface thereof facing the second electrode panel member to be inserted into the electrode-coupling hollow part, and the second electrode panel member has a second electrode coupling part protruding from a surface thereof facing the first electrode panel member to be inserted into the electrode-coupling hollow part and detachably coupled to the first electrode coupling part.

7: The cartridge of claim 6, wherein the first electrode coupling part and the second electrode coupling part are detachably connected to each other by screw connection.

8: An electrode cartridge coupling structure, comprising:
an electrode cartridge including an electrode part disposed to contact a patient's skin and apply an electric signal thereto; and
a cartridge coupling object provided with a cartridge coupling part into which a portion of the electrode cartridge is inserted and joined, and electrically connected to a control body that controls an operation of the electrode cartridge,
wherein the electrode cartridge is the electrode cartridge for skin treatment according to claim 1.

9: The structure of claim 8, further comprising:
a ball plunger part protruding from each side of the electrode cartridge; and
a ball insertion part provided on each inner side of the cartridge coupling part and into which the ball plunger part is inserted,
wherein the electrode cartridge is positioned so as to be rotatable around the ball plunger parts.

10: The structure of claim 9, wherein the ball plunger part and the ball insertion part share an electrical terminal structure that provides electrical connectivity therebetween so as to electrically connect the electrode part and the control body.

11: The structure of claim 8, further comprising:
an auxiliary electrode part located on an end side of the cartridge coupling part,
wherein the electrode part functions as an RF electrode having one polarity, and the auxiliary electrode part functions as an RF electrode having an opposite polarity.

12: The structure of claim 9, wherein the electrode cartridge is provided with a ring-shaped contact protrusion on each side thereof that surrounds the ball plunger part.

13: The structure of claim 12, wherein on the inner side of the cartridge coupling part, a cartridge stopper part is positioned to protrude, which catches the contact protrusion and prevents the electrode cartridge from being pushed and moved inward of the cartridge coupling part while in contact with the skin.

14: The structure of claim 13, wherein the cartridge stopper part is provided with a ball bearing part that rolls while in contact with the contact protrusion on a surface thereof facing the contact protrusion.

15: The structure of claim 9, further comprising:
a cartridge separation part positioned within the cartridge coupling part to be able to move forward and backward in a coupling direction of the electrode cartridge, and configured to push the electrode cartridge to separate the electrode cartridge from the cartridge coupling part.

16: The structure of claim 15, wherein the cartridge separation part comprises:
a pusher member positioned to be able to move forward and backward within the cartridge coupling part; and
a linear movement device positioned in the cartridge coupling object, and configured to move the pusher member forward and backward.

17: The structure of claim 16, wherein the cartridge separation part further comprises:
a manipulation switch part located on the cartridge coupling object to allow a practitioner to control an operation of the linear movement device.
